(19) United States
(12) Patent Application Publication
(10) Pub. No.: US 2025/0001169 A1
(43) Pub. Date: Jan. 2, 2025

(54) TRANSDUCER ARRAYS CAPABLE OF ASSUMING A SUBSTANTIALLY CONICAL SHAPE

(71) Applicant: Novocure GmbH, Root D4 (CH)

(72) Inventors: NOA HALAVEE, Tel Aviv (IL); Elie YAACOBI, Haifa (IL)

(73) Assignee: Novocure GmbH, Root D4 (CH)

(21) Appl. No.: 18/756,587

(22) Filed: Jun. 27, 2024

Related U.S. Application Data
(60) Provisional application No. 63/524,561, filed on Jun. 30, 2023.

(51) Int. Cl. A61N 1/36; A61N 1/04

(52) U.S. Cl. CPC A61N 1/36002; A61N 1/0408; A61N 1/0476; A61N 1/0496

(57) ABSTRACT

A transducer apparatus for delivering tumor treating fields to a subject's body, the transducer apparatus comprising: a substrate; and an array of at least one electrode disposed on the substrate, the array configured to be positioned over the subject's body with a face of the array facing the subject's body; wherein the transducer apparatus is substantially non-planar, wherein the transducer apparatus is substantially shaped as a truncated elliptical paraboloid, truncated oblique cone, or truncated cone, and a substantially circular opening is formed by an opening at a truncated portion of the truncated elliptical paraboloid, truncated oblique cone, or truncated cone.

### CROSS-REFERENCE TO RELATED APPLICATIONS

This application claims priority to U.S. Provisional Application No. 63/524,561, filed Jun. 30, 2023, which is incorporated herein by reference in its entirety. This application is related to U.S. Provisional Application No. 63/524,586, filed Jun. 30, 2023, which is incorporated herein by reference in its entirety.

### BACKGROUND

Tumor treating fields (TTFields) are low intensity alternating electric fields within the intermediate frequency range (for example, 50 kHz to 1 MHz), which may be used to treat tumors as described in U.S. Pat. No. 7,565,205. In current commercial systems, TTFields are induced non-invasively into the region of interest by electrode assemblies (e.g., arrays of capacitively coupled electrodes, also called electrode arrays, transducer arrays or simply “transducers”) placed on the patient's body and applying alternating current (AC) voltages between the transducers. Conventionally, a first pair of transducers and a second pair of transducers are placed on the subject's body. AC voltage is applied between the first pair of transducers for a first interval of time to generate an electric field with field lines generally running in the front-back direction. Then, AC voltage is applied at the same frequency between the second pair of transducers for a second interval of time to generate an electric field with field lines generally running in the right-left direction. The system then repeats this two-step sequence throughout the treatment.

### BRIEF DESCRIPTION OF THE DRAWINGS

FIGS. 1A, 1B, 2A, and 2B depict examples of transducers located on a subject's body for delivery of TTFields.

FIG. 3A depicts a top view of two example transducers.

FIG. 3B depicts a transducer of FIG. 3A applied to an example breast site.

FIG. 4A depicts a top view of two example transducers.

FIG. 4B depicts a transducer of FIG. 4A applied to an example breast site.

FIGS. 5A and 5B depict a top view of an example transducer.

FIGS. 5C and 5D depict a top view of an example transducer.

FIG. 6A depicts a top view of an example transducer.

FIG. 6B depicts the transducer of FIG. 6A applied to an example breast site.

FIG. 6C depicts a top view of an example transducer.

FIG. 6D depicts the transducer of FIG. 6C applied to an example breast site.

FIG. 7 depicts an example system to apply alternating electric fields to the subject's body.

FIG. 8 depicts an example computer apparatus.

FIG. 9 is a flowchart depicting an example method of determining shape and placement of transducers on a subject's body for applying TTFields.

### DESCRIPTION OF EMBODIMENTS

This application describes exemplary transducers (or transducer apparatuses) used to apply TTFields to a subject's body for treating one or more cancers.

Transducers used to apply TTFields to a subject's body often include multiple electrode elements electrically coupled together on a substrate and attached to the subject's body at a desired location, for example, via an adhesive backing of the substrate or a separately applied adhesive. Transducers may have large, rectangular surfaces so as to maximize a number of electrode elements that are located on the transducer for applying TTFields to the subject's body. As recognized by the inventors, such transducers may not desirably attach to portions of a subject having a non-planar surface, such as a breast.

The inventors have now recognized that a need exists for transducers that can conform to three-dimensional structures of a subject's body. For example, transducers may be structured to surround, conform, adapt to, or be positioned on or around a breast of a subject and/or a chemotherapy port of a subject. In some embodiments described herein, transducers may be capable of being deformed from being substantially planar to being substantially conical, such as a substantially truncated elliptical paraboloid, a substantially truncated oblique cone, or a substantially truncated cone, or the like. When situated around a breast site, for example, a substantially circular opening may be formed by an opening at a truncated portion of the truncated elliptical paraboloid, truncated oblique cone, or truncated cone, for example, to avoid coverage of a subject's nipple. When situated around a chemotherapy port, for example, the chemotherapy port may be located within an opening of the transducer, where the transducer may have a single part or a plurality of parts to form the opening.

The system described herein further provides a practical method to determine a shape and placement of transducers on a subject's body for applying tumor treating fields. For example, a computer-based system may select a first location on the subject's body for placement of a first transducer and a second location on the subject's body for placement of a second transducer, based on one or more simulations of an electric field distribution through a target region in the subject's body. The computer-based system may select a transducer such as described herein as one of the selected transducers.

Different types of transducers are described herein. Each of the embodiments disclosed herein may be used for one or more of the transducer types described herein.

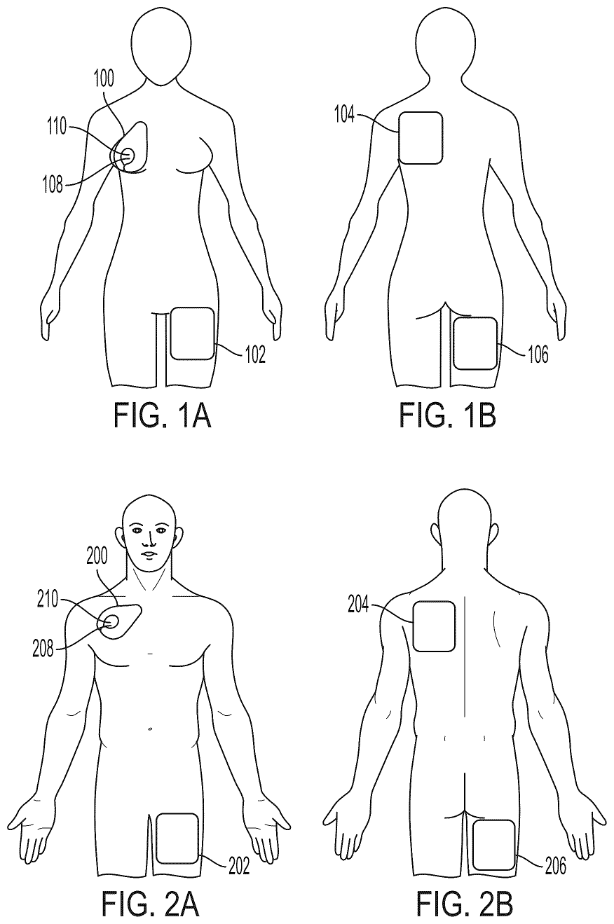

FIGS. 1A and 1B depict an example of transducers positioned at locations on a subject's body for delivery of TTFields. FIG. 1A depicts a first transducer 100 located on the front of the subject's right breast and a second transducer 102 located on the front of the subject's left thigh. FIG. 1B depicts a third transducer 104 located on the left side of the subject's upper back and a fourth transducer 106 located on the back of the subject's right thigh. Each of the transducers 100, 102, 104, and 106 may include one or more electrode elements located on a surface that is flexible for contouring the transducer to the subject's body. The transducers 100, 102, 104, and 106 may be capable of delivering TTFields to the subject's body.

Similarly, FIGS. 2A and 2B depict another example of transducers positioned at locations on a subject's body for delivery of TTFields. FIG. 2A depicts a first transducer 200 located on the front of the subject's right thorax and a second transducer 202 located on the front of the subject's left thigh. FIG. 2B depicts a third transducer 204 located on the left side of the subject's upper back and a fourth transducer 206 located on the back of the subject's right thigh. Each of the 200, 202, 204, and 206 may include one or more electrode elements located on a surface that is flexible for contouring the transducer to the subject's body.

Transducers arranged on a subject's torso (as shown in FIGS. 1A-2B) are capable of applying TTFields to a tumor in the subject's thorax or abdomen. The transducers may be located at various other combinations of locations on the subject's torso than those of FIGS. 1A-2B.

FIGS. 1A and 1B and FIGS. 2A and 2B illustrate an assembly for applying TTFields to a subject's body while avoiding at least one area with an anatomic feature or device. For example, in FIG. 1A, the surface of the transducer 100 is shaped and adapted for contouring over a breast of the subject's body while avoiding a nipple 108 of the subject's body. In some embodiments, once placed on the subject's body, a substantially circular opening 110 coincides with the nipple 108 of the subject's body, such that no electrodes of the transducer are located over the nipple 108.

As another example, in FIG. 2A, the surface of the transducer 200 is shaped and adapted for contouring to avoid a chemotherapy port 208 on the subject's body. In particular, the surface of the transducer 200 is adapted to be positioned on the subject's body such that a substantially circular opening 210 of the transducer 200 coincides with a location on the subject's body having the chemotherapy port 208. In another example, the surface of the transducer 200 may be positioned on the subject's body with two opposing portions of the transducer surface spaced apart to straddle a location on the subject's body having the chemotherapy port 208. No electrodes of the transducer 200 may be located over the chemotherapy port 208. Chemotherapy ports 208 are often inserted into a subject's body prior to the subject receiving TTFields treatment. The transducers disclosed herein may enable the application of TTFields to a region of interest in the subject's thorax or abdomen without interfering with or being affected by the subject's chemotherapy port 208.

Turning back to FIGS. 1A and 1B, one or more other transducers 102, 104, and 106 may have a different shape than the transducer 100. As illustrated, for example, each of the second, third, and fourth transducers 102, 104, and 106 of the assembly has a different shape than the first transducer 100. In some embodiments, each of the second, third, and fourth transducers 102, 104, and 106 may have the same or a substantially similar shape to each other. As illustrated, the surface of at least one of the transducers 102, 104, and 106 may have a substantially convex shape. More particularly, the surface of at least one of the transducers 102, 104, and 106 may have a rectangular, substantially rectangular with rounded corners (as illustrated), circular, oval, ovaloid, ovoid, or elliptical shape. Similar situations may apply to the transducers 200, 202, 204, and 206. In particular, the transducer 200 illustrated in FIG. 2A may have a similar shape as the transducer 100 illustrated in FIG. 1A, and the transducers 202, 204, and 206 illustrated in FIGS. 2A and 2B may have similar shapes as the transducers 102, 104, and 106 shown in FIGS. 1A and 1B.

In other embodiments, one or more of the other transducers 102, 104, and 106 may have a surface having the same shape or a mirror image shape of the transducer 100 of FIGS. 1A and 1B. Similarly, one or more of the other transducers 202, 204, and 206 may have a surface having the same shape or a mirror image shape compared to the transducer 200 of FIGS. 2A and 2B.

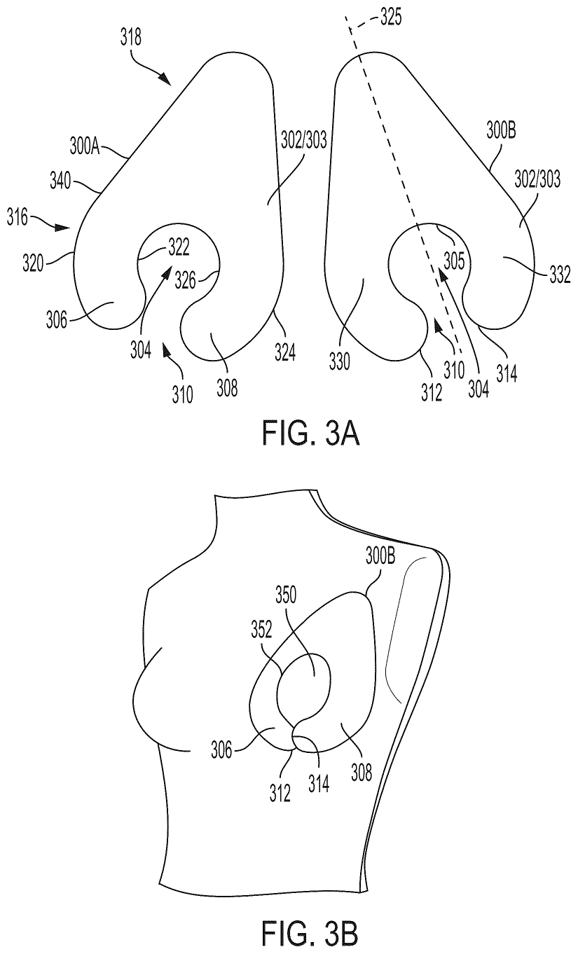

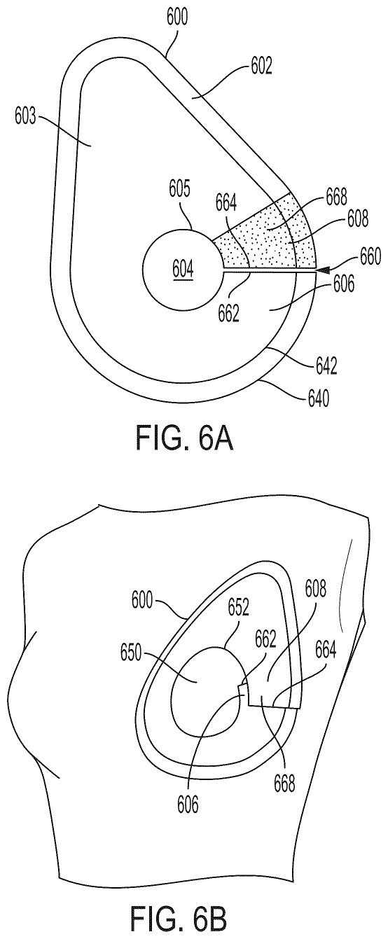

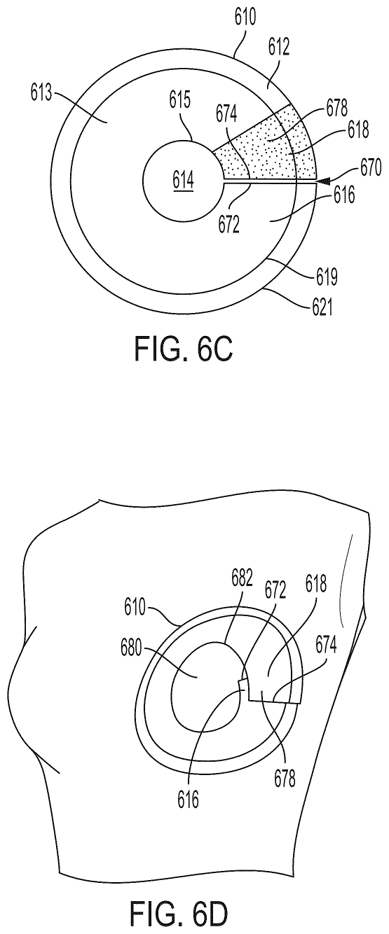

FIG. 3A depicts a top view of two example transducers 300A and 300B. The transducer 300A, 300B (and any other transducers disclosed or discussed herein) may be capable of delivering TTFields to a subject's body. The transducers 300A and 300B have the same shape and the same features. The transducer 300A, 300B may include a substrate 302 (having a first side to face the subject and a second side opposite the first side) and an array of at least one electrode (not shown) disposed on the first side of the substrate 302. In some embodiments, the transducer 300A, 300B (and any other transducers disclosed or discussed herein) may include a layer of anisotropic material, as discussed further herein below. For example, the layer of anisotropic material may overlay the at least one electrode on the skin-facing side of the array of the at least one electrode (such that the layer of anisotropic material may be facing the first side of the substrate 302). In some embodiments, the layer of anisotropic material may be present as, or may compromise, a laminate having a layer of conductive adhesive, a layer of anisotropic material, and a layer of conductive adhesive. In some embodiments, the anisotropic material is a sheet of graphite. In some embodiments, the layer of anisotropic material is a sheet of pyrolytic graphite, graphitized polymer film, or graphite foil made from compressed high purity exfoliated mineral graphite. In some embodiments, the layer of anisotropic material may take the same or similar shape as that of the substrate 302, and may be of a similar size or slightly smaller than that of the substrate. Moreover, in some embodiments, a layer of adhesive may be present between the substrate and the layer of anisotropic material. For simplicity, FIGS. 1-5 do not show both the substrate and the layer of anisotropic material, although both may be present in one or more (or all) of these embodiments. For embodiments without a layer of anisotropic material, the shape of the main body of the transducer may (or may not) reflect the shape of the substrate (e.g., 302, 402, 502, 702 in FIGS. 3-5 and 7). FIG. 6A shows the layer of anisotropic material as 603, the exterior edge 642 of which may (as in FIG. 6A), or may not, contour the shape of the main body of the transducer. Similarly, in describing the process of deforming the planar transducer to produce the non-planar transducer, it is to be understood that the substrate and the layer of anisotropic material may have the same or similar shape (overlapping one on the other) and may be adhered to one another. Accordingly, in some embodiments, folding a first portion of the substrate over a second portion of the substrate may include folding both layers (the substrate and the layer of anisotropic material) simultaneously.

The transducer 300A, 300B (and any other transducers disclosed or discussed herein) may include any of the features discussed herein and may include any desired number of electrode elements (e.g., one or more electrode elements). The transducer 300A, 300B may be configured to be positioned over a subject's body with a face of the array facing the subject's body. In some embodiments, the transducer 300A, 300B may be substantially planar. In some embodiments, the transducer 300A, 300B may be substantially planar prior to being located, applied, or affixed to a subject. In some embodiments, when viewed from a direction perpendicular to a face of the array, the transducer 300A, 300B may have a substantially pear-shaped or rounded triangular-shaped surface. In some embodiments, when viewed from a direction perpendicular to the face of the array, the substrate 302 (or layer of anisotropic material 303) may have a substantially pear-shaped or rounded triangular-shaped surface.

In some embodiments, when viewed from a direction perpendicular to the face of the array and when the transducer 300A, 300B is substantially planar, the substrate 302 (or layer of anisotropic material 303) may have an opening 304 located towards a wider portion 316 than a narrower portion 318 of the substrate 302 (or layer of anisotropic material 303). In some embodiments, no electrodes are in the opening 304. The opening 304 may have at least one concave edge 305 defining the opening 304 between two opposing portions, namely a first portion 330 and a second portion 332, of the substrate 302 (or layer of anisotropic material 303). The concave edge 305 may include a substantially C-shaped concave surface. The first portion 330 and the second portion 332 may be mirror images of each other and may have reflectional symmetry. The reflectional symmetry of the first portion 330 and the second portion 332 may be about a centerline 325. In other embodiments, the first portion 330 and the second portion 332 may not be mirror images of each other and may not have reflectional symmetry.

In some embodiments, when viewed from a direction perpendicular to the face of the array and when the transducer 300A, 300B is substantially planar, the transducer 300A, 300B may include a first end portion 306 separated from a second end portion 308 by a gap 310. The gap 310 may be located closer to the wider portion 316 than the narrower portion 318 of the substrate 302 (or layer of anisotropic material 303). The centerline 325 may run through a longest dimension of the substrate 302 (or layer of anisotropic material 303) and through a center of the gap 310. The substrate 302 (or layer of anisotropic material 303) may have reflectional symmetry, and the reflectional symmetry of the substrate 302 (or layer of anisotropic material 303) may be about the centerline 325.

The substrate 302 (or layer of anisotropic material 303) may have two opposing ends (or opposing sides), namely a first end 312 and a second end 314. The gap 310 may be defined as being between the two opposing ends 312, 314 of the substrate 302 (or layer of anisotropic material 303). The first end 312 may include a convex edge, and the second end 314 may include a convex edge.

The first end portion 306 may have a first edge 320 and a second edge 322. The first edge 320 may define a portion of an exterior edge 340 of the substrate 302 (or layer of anisotropic material 303) and may be convex shaped. The second edge 322 may define a portion of the concave edge 305 of the opening 304 and may be concave shaped. The second end portion 308 may have a first edge 324 and a second edge 326. The first edge 324 may define a portion of the exterior edge 340 of the substrate 302 (or layer of anisotropic material 303) and may be convex shaped. The second edge 326 may define a portion of the concave edge 305 of the opening 304 and may be concave shaped. The opening 304 may have a partially circular, nearly circular, substantially partially circular, or substantially nearly circular edge defined by the first edge 322 and the second edge 326.

In some embodiments, the first portion 330 and the second portion 332 may be used to define the opening 304. In some embodiments, the first end portion 306 and the second end portion 308 may be used to define the opening 304. In some embodiments, the first end portion 306 and the second end portion 308 may be used to define the gap 310. In some embodiments, the first portion 330 and the second portion 332 may be closer to the narrower portion 318, and the first end portion 306 and the second end portion 308 may be closer to the wider portion 316. In some embodiments, the first portion 330 and the first end portion 306 may at least partially overlap, and in some embodiments, the second portion 332 and the second end portion 308 may at least partially overlap.

In some embodiments, the first end portion 306 and the second end portion 308 may each include a portion of the array of at least one electrode. In other embodiments, only one of the first end portion 306 or the second end portion 308 includes a portion of the array of at least one electrode. In some embodiments, the first end portion 306 and the second end portion 308 are part of a single continuous substrate 302 (or layer of anisotropic material 303). In other embodiments, the first end portion 306 and the second end portion 308 are located on two separate discontinuous sections of substrate 302 (or layer of anisotropic material 303).

The transducer 300A, 300B may be capable of being deformed from being substantially planar to being substantially non-planar, such as being deformed to be shaped as a substantially truncated elliptical paraboloid or substantially truncated oblique cone, or the like. As a non-planar shape, the transducer 300A, 300B may be substantially conical, such as substantially truncated elliptical paraboloid or substantially truncated oblique cone.

FIG. 3B depicts the transducer 300B applied to an example breast site. For ease in explanation, the transducer 300B is depicted on a mannequin. When the transducer 300B is deformed and substantially shaped as a truncated elliptical paraboloid or truncated oblique cone, a substantially circular opening 350 may be formed by the opening 304 at a truncated portion 352 of the truncated elliptical paraboloid or truncated oblique cone. In some embodiments, when the transducer 300B is substantially shaped as a truncated elliptical paraboloid or truncated oblique cone, the substantially circular opening 350 may be formed at the truncated portion 352 of the truncated elliptical paraboloid or truncated oblique cone by removing the gap 310 between two opposing ends, namely between the first end 312 and the second end 314 of the substrate 302 (or layer of anisotropic material 303). In some embodiments, when the transducer 300B is substantially shaped as a truncated elliptical paraboloid or truncated oblique cone, the first end portion 306 abuts, touches, or overlaps the second end portion 308, and/or the second end portion 308 abuts, touches, or overlaps the first end portion 306. As shown in FIG. 3B, the second end 314 of the second end portion 308 overlaps the first end 312 of the first end portion 306. As shown in FIG. 3B, the abutting, touching, or overlapping of the first end portion 306 and the second end portion 308 is situated at a lower portion of the breast of the subject. A similar description may apply for the transducer 300A (with the same labelling notations, shown in FIG. 3A/3B) when the transducer 300A is deformed and substantially shaped as a truncated elliptical paraboloid or truncated oblique cone. Transducer 300A may similarly be applied to a breast site.

In some embodiments, the transducer 300A, 300B may be substantially non-planar. In such embodiments, the transducer 300A, 300B may be substantially shaped as a truncated elliptical paraboloid or truncated oblique cone, and a substantially circular opening 350 may be formed by an opening 304 at a truncated portion 352 of the truncated elliptical paraboloid or truncated oblique cone. In such embodiments, the transducer 300A, 300B may be adapted to be positioned on or around an anatomical feature of a subject, for example a breast (FIG. 3B). The substantially circular opening 350 may coincide with a first location on a subject, for example a nipple, and no electrodes of the transducer array may be located over the nipple.

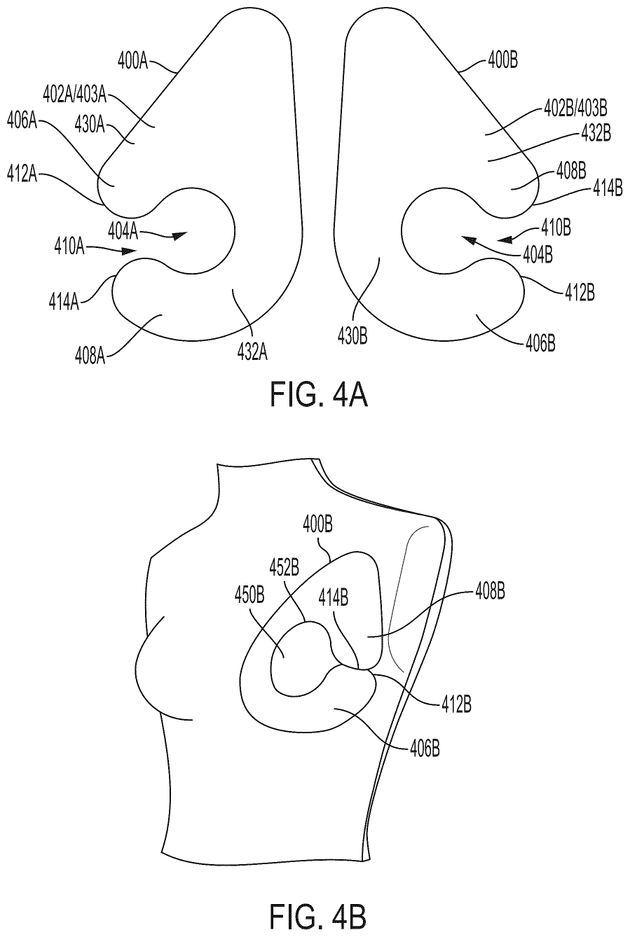

FIG. 4A depicts a top view of two example transducers 400A and 400B. The transducers 400A and 400B are mirror images of each other, but otherwise have the same shape and the same features. Although, in FIG. 4A the two example transducers 400A and 400B are shown as mirror images of each other, they need not be, and, accordingly, the labelling system in FIG. 4A follows a left-side/right-side convention as opposed to a mirror image convention. The transducers 400A and 400B are similar to the transducer 300A, 300B (and follow a similar description and labeling notation) but differ in that each of the transducers 400A and 400B do not have reflectional symmetry. The substrates 402A, 402B (or layer of anisotropic material 403A, 403B) have openings 404A, 404B and gaps 410A, 410B respectively. In some embodiments, the gaps 410A, 410B may be situated on one side of the substrate 402A, 402B (or layer of anisotropic material 403A, 403B). When viewed from a direction perpendicular to the face of the array and when the transducer 400A is substantially planar, the gap 410A is on the left side of the substrate 402A (or layer of anisotropic material 403A). The first portion 430A is smaller than the second portion 432A. When viewed from a direction perpendicular to the face of the array and when the transducer 400B is substantially planar, the gap 410B is on the right side of the substrate 402B (or layer of anisotropic material 403B). The first portion 430B is larger than the second portion 432B.

The transducer 400A, 400B may be capable of being deformed from being substantially planar to being substantially non-planar, such as being deformed to be shaped as a truncated elliptical paraboloid or truncated oblique cone. FIG. 4B depicts the transducer 400B applied to an example breast site. For ease in explanation, the transducer 400B is depicted on a mannequin. When the transducer 400B is deformed and substantially shaped as a truncated elliptical paraboloid or truncated oblique cone, a substantially circular opening 450B may be formed by the opening 404B at a truncated portion 452B of the truncated elliptical paraboloid or truncated oblique cone. In some embodiments, when the transducer 400B is substantially shaped as a truncated elliptical paraboloid or truncated oblique cone, the substantially circular opening 450B may be formed at the truncated portion 452B of the truncated elliptical paraboloid or truncated oblique cone by removing the gap 410B between two opposing ends, namely between the first end 412B and the second end 414B of the substrate 402B (or layer of anisotropic material 403B). In some embodiments, when the transducer 400B is substantially shaped as a truncated elliptical paraboloid or truncated oblique cone, the first end portion 406B abuts, touches, or overlaps the second end portion 408B, and/or the second end portion 408B abuts, touches, or overlaps the first end portion 406B. As shown in FIG. 4B, the second end 414B of the second end portion 408B overlaps the first end 412B of the first end portion 406B. As shown in FIG. 4B, the abutting, touching, or overlapping of the first end portion 406B, and the second end portion 408B is situated at a lower portion of the breast of the subject (although other positions of the abutting, touching, or overlapping may be present in other embodiments). A similar description may apply for the transducer 400A (with similar labelling notations shown in FIG. 4A, except that for transducer 400A, the first end portion is 408A and the second end portion is 406A; and the second end is 412A and the first end is 414A) when the transducer 400A is deformed and substantially shaped as a truncated elliptical paraboloid or truncated oblique cone. Transducer 400A may similarly be applied to a breast site.

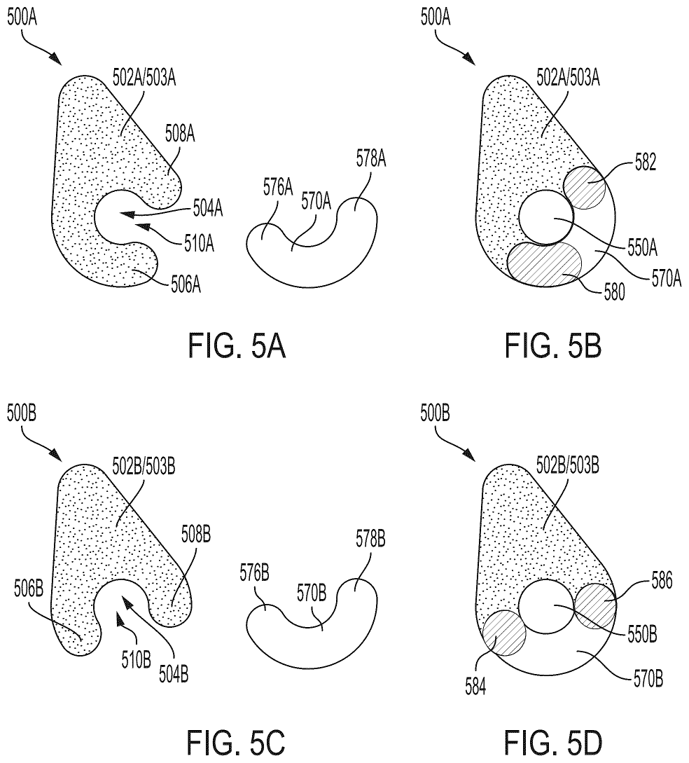

FIGS. 5A and 5B depict a top view of an example transducer 500A. The transducer 500A is similar to the transducer 400B but includes a second substrate (and/or layer of anisotropic material) 570A. In some embodiments, the transducer 500A may include a second substrate (and/or layer of anisotropic material) 570A separate from the substrate 502A (or layer of anisotropic material 503A) (FIG. 5A). A second array of at least one electrode may be disposed on the second substrate (and/or layer of anisotropic material) 570A, and the second array may be configured to be positioned over the subject's body with a face of the second array facing the subject's body. In some embodiments, the second substrate (and/or layer of anisotropic material) 570A is substantially C-shaped. In some embodiments, the transducer 500A may include the second substrate (and/or layer of anisotropic material) 570A which may abut, touch or overlap the first substrate 502A (or layer of anisotropic material 503A) (FIG. 5B). In some embodiments, when the transducer 500A (which may include abutting, touching or overlapping second substrate (and/or layer of anisotropic material) 570A) is substantially shaped as a truncated elliptical paraboloid or truncated oblique cone, a substantially circular opening 550A is defined by the former opening 504A (within the former gap 510A, FIG. 5A) and the substantially C-shaped second substrate (and/or layer of anisotropic material) 570A. In some embodiments, when the transducer 500A is substantially shaped as a truncated elliptical paraboloid or truncated oblique cone, a first end portion 576A of the substantially C-shaped second substrate (and/or layer of anisotropic material) 570A may overlap the first end portion 506A of the substrate 502A (or layer of anisotropic material 503A) in an overlap area 580 (shaded area in FIG. 5B), and a second end portion 578A of the substantially C-shaped second substrate (and/or layer of anisotropic material) 570A may overlap the second end portion 508A of the substrate 502A (or layer of anisotropic material 503A) in an overlap area 582 (shaded area in FIG. 5B). In FIG. 5B the overlap area 580 and the overlap area 582 have a different size and/or a different shape. However, in some embodiments, the overlap area 580 and the overlap area 582 may have a same size and/or a same shape. In some embodiments, the array disposed on substrate 502A and the second array disposed on the substrate 570A are electrically connected when one overlaps (and contacts) the other. Prior to locating the transducer 500A on a subject, the substrate 502A (or layer of anisotropic material 503A) and the second substrate (and/or layer of anisotropic material) 570A may be separate from each other (and not electrically connected), as shown in FIG. 5A. Once the transducer 500A is located on a subject, the substrate 502A (or layer of anisotropic material 503A) and the second substrate (and/or layer of anisotropic material) 570A may be abutting, touching, or overlapping each other, as shown in FIG. 5B. In alternative embodiments, the substrate 502A (or layer of anisotropic material 503A) and the second substrate (and/or layer of anisotropic material) 570A may be abutting, touching, or overlapping each other prior to positioning the transducer 500A on the subject.

FIGS. 5C and 5D depict a top view of an example transducer 500B. The transducer 500B is similar to the transducer 300A, 300B but, in addition to the substrate 502B (or layer of anisotropic material 503B) (analogous to the substrate 302/layer of anisotropic material 303), the transducer 500B includes a second substrate (and/or layer of anisotropic material) 570B. Similar to the second substrate (and/or layer of anisotropic material) 570A, the second substrate (and/or layer of anisotropic material) 570B may also be substantially C-shaped. The substrate 502B (or layer of anisotropic material 503B) of the transducer 500B may be substantially similar in structure to the substrate 502A (or layer of anisotropic material 503A) of the transducer 500A except that the gap 510B of the opening 504B may be situated in a different exemplary position (FIG. 5C) as compared to the gap 510A of opening 504A (FIG. 5A). In some embodiments, when the transducer 500B is substantially shaped as a truncated elliptical paraboloid or truncated oblique cone, a first end portion 576B of the substantially C-shaped second substrate (and/or layer of anisotropic material) 570B may overlap a first end portion 506B of the substrate 502B (or layer of anisotropic material 503B) in an overlap area 584 (shaded area in FIG. 5D), and a second end portion 578B of the substantially C-shaped second substrate (and/or layer of anisotropic material) 570B may overlap a second end portion 508B of the substrate 502B (or layer of anisotropic material 503B) in an overlap area 586 (shaded area in FIG. 5D). In FIG. 5D the overlap area 584 and the overlap area 586 have a same size and/or a same shape (although they need not be; in some embodiments, the overlap area 584 and the overlap area 586 may have a different size and/or a different shape). In other respects, the formation and operation of the transducer 500B (including formation of a substantially circular opening 550B) may be similar to the transducer 500A (including formation of a substantially circular opening 550A) and may be similarly shaped as a truncated elliptical paraboloid or truncated oblique cone. In some embodiments, the array disposed on substrate 502B and the second array disposed on the substrate 570B are electrically connected when one overlaps (and contacts) the other. Prior to locating the transducer 500B on a subject, the substrate 502B (or layer of anisotropic material 503B) and the second substrate (and/or layer of anisotropic material) 570B may be separate from each other (and not electrically connected), as shown in FIG. 5C. Once the transducer 500B is located on a subject, the substrate 502B (or layer of anisotropic material 503B) and the second substrate (and/or layer of anisotropic material) 570B may be abutting, touching, or overlapping each other, as shown in FIG. 5D. In alternative embodiments, the substrate 502B (or layer of anisotropic material 503B) and the second substrate (and/or layer of anisotropic material) 570B may be abutting, touching, or overlapping each other prior to positioning the transducer 500B on the subject.

When applied to an anatomical feature of a subject, such as a breast or a chemotherapy port, a transducer may be difficult to fit in the correct position. For example, the transducer may be too small to cover a correct location on a breast; or, as another example, it may not be possible or practical to place the transducer on the chemotherapy port. In such situations, for example, transducers 500A, 500B may be used by first positioning the substrate 502A, 502B (or layer of anisotropic material 503A, 503B) on the subject, and then positioning the second substrate (and/or layer of anisotropic material) 570A, 570B on the subject to form a truncated elliptical paraboloid or truncated oblique cone having a substantially circular opening 550A, 550B. In some embodiments, the second substrate (and/or layer of anisotropic material) 570A and 570B may have the same shape and size, or very similar shape and/or size, and may be capable of being used with different shaped substrates, such as the substrate 502A and the substrate 502B (or layer of anisotropic material 503A, 503B).

FIG. 6A depicts a top view of an example transducer 600. The transducer 600 is similar to the example transducer 300A, 300B in FIG. 3 and with similar feature labeling, but instead of having a gap 310, the transducer 600 includes a slit 660. The top view in FIG. 6A additionally depicts a layer of anisotropic material 603 which may overlay (and partially obscure) a substrate 602. The substrate 602 may be, or may comprise, an adhesive bandage to affix the transducer 600 to the subject. When viewed from a direction perpendicular to the face of the transducer 600 and when the transducer 600 is substantially planar, the slit 660 may be located between an exterior edge 640 of the substrate 602 and a concave edge 605 of the opening 604. The slit 660 may separate a first end portion 606 of the transducer 600 from a second end portion 608 of the transducer 600. The slit 660 may be defined by a first edge 662 of the first end portion 606 and a second edge 664 of the second end portion 608. As an example, the slit 660 may be a visible gap between the first edge 662 and the second edge 664. As an example, the slit 660 may be formed by scoring the transducer 600. The first edge 662 and the second edge 664 may both be straight lines between the exterior edge 640 of the substrate 602 and the concave edge 605. The slit 660 may be through the layer of anisotropic material 603 having a peripheral edge 642. The slit 660 may be through the substrate 602 having the exterior edge 640. The slit 660 may be through both the substrate 602 and the layer of anisotropic material 603 (as shown in FIG. 6A). Similar embodiments may exist for transducers having a substrate but no layer of anisotropic material, or having a layer of anisotropic material but no substrate.

FIG. 6B depicts the transducer 600 applied to an example breast site. For ease in explanation, the transducer 600 is depicted on a mannequin. In some embodiments, when the transducer 600 is deformed and substantially shaped as a truncated elliptical paraboloid or truncated oblique cone, a substantially circular opening 650 may be formed by the opening 604 at a truncated portion 652 of the truncated elliptical paraboloid or truncated oblique cone. To form the substantially circular opening 650, the first end portion 606 may overlap the second end portion 608 (or vice-versa). Referring to FIG. 6A, an overlap portion 668 (the shaded section) of the second end portion 608 may be designated to overlap the first end portion 606. In FIG. 6B, the second end portion 608 overlaps at least a part of the first end portion 606. In other embodiments, the first end portion 606 may overlap at least a part of the second end portion 608. Referring back to FIG. 6B, the edge 664 at least partially overlaps (or extends onto) the first end portion 606, and the edge 662 is at least partially behind the second end portion 608. Due to the overlapping, the substantially circular opening 650 may or may not be perfectly circular (the latter is illustrated in FIG. 6B).

FIG. 6C depicts a top view of an example transducer 610. The transducer 610 is similar to the example transducer 600 in FIG. 6A and with similar feature labeling, but instead of having a substantially pear-shaped or rounded triangular-shaped surface, the transducer 610 has a circular shaped surface. With a circular shaped surface, the transducer 610 may be better adapted than a substantially pear-shaped or rounded triangular-shaped surface to be placed on or around a breast of a subject or a chemotherapy port of a subject. The top view in FIG. 6C additionally depicts a layer of anisotropic material 613 which may overlay (and partially obscure) a substrate 612. The substrate 612 may be, or may comprise, an adhesive bandage to affix the transducer 610 to the subject. When viewed from a direction perpendicular to the face of the array and when the transducer 610 is substantially planar, a slit 670 may be located between an exterior edge 621 of the substrate 612 and a concave edge 615 of the opening 614. The slit 670 may separate a first end portion 616 of the transducer 610 from a second end portion 618 of the transducer 610. The slit 670 may be defined by a first edge 672 of the first end portion 616 and a second edge 674 of the second end portion 618. As an example, the slit 670 may be a visible gap between the first edge 672 and the second edge 674. As an example, the slit 670 may be formed by scoring the transducer 610. The first edge 672 and the second edge 674 may both be straight lines between the exterior edge 621 of the substrate 612 and the concave edge 615. The slit 670 may be through the layer of anisotropic material 613 having peripheral edge 619. The slit 670 may be through the substrate 612 having exterior edge 621. The slit 670 may be through both the substrate 612 and the layer of anisotropic material 613 (as shown in FIG. 6C). Similar embodiments may exist for transducers having a substrate but no layer of anisotropic material, or having a layer of anisotropic material but no substrate.

FIG. 6D depicts the transducer 610 applied to an example breast site. For ease in explanation, the transducer 610 is depicted on a mannequin. In some embodiments, when the transducer 610 is deformed and substantially shaped as a truncated cone, a substantially circular opening 680 may be formed by the opening 614 at a truncated portion 682 of the truncated cone. To form the substantially circular opening 680, the first end portion 616 may overlap the second end portion 618 (or vice-versa). Referring to FIG. 6C, an overlap portion 678 (the shaded section) of the second end portion 618 may be designated to overlap the first end portion 616. In FIG. 6D, the second end portion 618 overlaps at least a part of the first end portion 616 (by an amount equivalent to the overlap portion 678). In other embodiments, the first end portion 616 may overlap at least a part of the second end portion 618. Referring back to FIG. 6D, the edge 674 at least partially overlaps (or extends onto) the first end portion 616, and the edge 672 is at least partially behind the second end portion 618. Due to the overlapping, the substantially circular opening 680 may (should be) perfectly circular or may not be perfectly circular (the latter is illustrated in FIG. 6D, primarily to illustrate the first end portion 616 and the first edge 672, both of which would be obscured if the overlap were perfect).

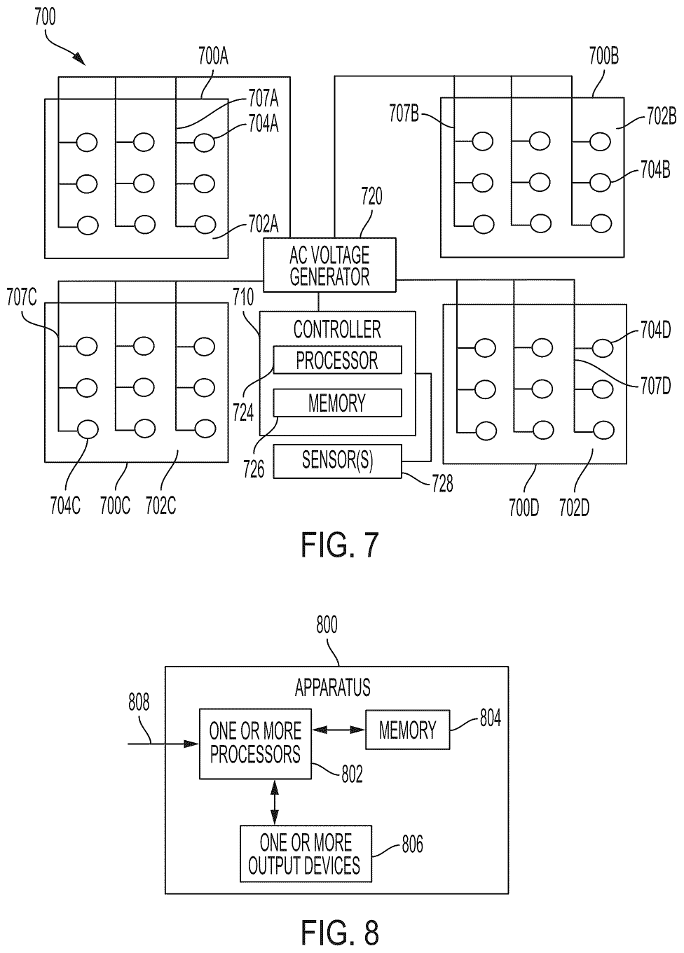

FIG. 7 depicts an example apparatus 700 to apply alternating electric fields (e.g., TTFields) to the subject's body. The system may be used for treating a target region of a subject's body with an alternating electric field. In an example, the target region may be in the subject's torso, and an alternating electric field may be delivered to the subject's body via two pairs of transducer arrays positioned on at least one of a thorax, an abdomen, or one or both thighs of the subject's body (such as, for example, in FIGS. 1A and 1B, which has four transducers 100, 102, 104, and 106, and in FIGS. 2A and 2B, which has four transducers 200, 202, 204, and 206). In another example, the target region may be in the subject's brain, and an alternating electric field may be delivered to the subject's body via two pairs of transducer arrays positioned on a head of the subject's body. Other transducer array placements on the subject's body may be possible.

The example apparatus 700 depicts an example system having four transducers (or “transducer arrays”) 700A-D. Each transducer 700A-D may include substantially flat electrode elements 704A-D positioned on a substrate 702A-D and electrically and physically connected (e.g., through conductive wiring 707A-D). The substrates 702A-D may include, for example, cloth, foam, flexible plastic, and/or conductive medical gel or adhesive. As described herein, the transducers 700A-D may be substantially planar (flat), but one or more of the transducers 700A-D may be configured such that the transducer is capable of being deformed from being substantially planar to being substantially non-planar (e.g., being substantially shaped as a truncated elliptical paraboloid, a truncated oblique cone, a truncated cone, or the like). Two transducers (e.g., 700A and 700D) may be a first pair of transducers configured to apply an alternating electric field to a target region of the subject's body. The other two transducers (e.g., 700B and 700C) may be a second pair of transducers configured to similarly apply an alternating electric field to the target region.

The transducers 700A-D may be coupled to an AC voltage generator 720, and the system may further include a controller 710 communicatively coupled to the AC voltage generator 720. The controller 710 may include a computer having one or more processors 724 and memory 726 accessible by the one or more processors. The memory 726 may store instructions that when executed by the one or more processors control the AC voltage generator 720 to induce alternating electric fields between pairs of the transducers 700A-D according to one or more voltage waveforms and/or cause the computer to perform one or more methods disclosed herein. The controller 710 may monitor operations performed by the AC voltage generator 720 (e.g., via the processor(s) 724). One or more sensor(s) 728 may be coupled to the controller 710 for providing measurement values or other information to the controller 710 (e.g., thermistors providing temperature measurements).

The voltage generator 720 may provide one or more voltages to the different pairs of transducers (e.g., 700A/D, 700B/C) for applying alternating electric fields to the subject's body. The controller 710 may instruct the voltage generator 720 to generate the one or more voltages according to one or more waveforms. For example, the method described below with reference to FIG. 7 may be implemented using the voltage generator 720 and controller 710.

The structure of the transducers 700A-D may take many forms. The transducers may be affixed to the subject's body or attached to or incorporated in clothing covering the subject's body. The transducer may include suitable materials for attaching the transducer to the subject's body. For example, the suitable materials may include cloth, foam, flexible plastic, and/or a conductive medical gel.

The transducer may include any desired number of electrode elements (e.g., one or more electrode elements). For example, the transducer may include one, two, three, four, five, six, seven, eight, nine, ten, or more electrode elements (e.g., twenty electrode elements). Various shapes, sizes, and materials may be used for the electrode elements. Any constructions for implementing the transducer (or electric field generating device) for use with embodiments of the invention may be used as long as they are capable of (a) delivering TTFields to the subject's body and (b) being positioned at the locations specified herein. The transducer may be conductive or non-conductive. In some embodiments, an AC signal may be capacitively coupled into the subject's body. In some embodiments, at least one electrode element of the first, the second, the third, or the fourth transducer can include at least one ceramic disk that is adapted to generate an alternating electric field. In some embodiments, at least one electrode element of the first, the second, the third, or the fourth transducer includes a polymer film that is adapted to generate an alternating field. For example, at least one electrode element may include polymer films disposed over pads on a printed circuit board or over substantially planar pieces of metal. In an embodiment, such polymer films have a high dielectric constant, such as, for example, a dielectric constant greater than 10.

The disclosed transducers may also include a layer of anisotropic material located on a side of the array of electrode elements facing the subject's body, as disclosed, for example, in United States Patent Application Publication No. 2023/0037806 A1. The layer of anisotropic material may have anisotropic thermal properties and/or anisotropic electrical properties. If the layer of anisotropic material has anisotropic thermal properties (for example, greater thermal conductivity in the plane of the layer than through the plane of the layer), then the layer spreads heat out more evenly over a larger surface area. If the layer of anisotropic material has anisotropic electrical properties (for example, greater electrical conductivity in the plane of the layer than through the plane of the layer), then the layer spreads the current out more evenly over a larger surface area. In each case, this lowers the temperature of any hot spots and raises the temperature of the cooler regions when a given AC voltage is applied to the array of electrode elements. Accordingly, the current can be increased (thereby increasing the therapeutic effect) without exceeding the safety temperature threshold at any point on the subject's skin.

In some embodiments, the layer of anisotropic material is anisotropic with respect to electrical conductivity properties. In some embodiments, the layer of anisotropic material is anisotropic with respect to thermal conductivity properties. In some preferred embodiments, the layer of anisotropic material is anisotropic with respect to both electrical conductivity properties and thermal conductivity properties.

The anisotropic thermal properties include directional thermal properties. Specifically, the layer of anisotropic material may have a first thermal conductivity in a direction that is perpendicular to its front face (skin-facing surface) that is different from a thermal conductivity of the layer of anisotropic material in directions that are parallel to the front face. For example, the thermal conductivity of the layer of anisotropic material in directions parallel to the front face is more than two times higher than the first thermal conductivity. In some preferred embodiments, the thermal conductivity in the parallel directions is more than ten times higher than the first thermal conductivity. For example, the thermal conductivity of the sheet in directions that are parallel to the front face may be more than: 1.5 times, 2 times, 3 times, 5 times, 10 times, 20 times, 100 times, 200 times, or even more than 1,000 times higher than the first thermal conductivity.

The anisotropic electrical properties include directional electrical properties. Specifically, the layer of anisotropic material may have a first electrical conductivity (or, conversely, resistance) in a direction that is perpendicular to its front face that is different from an electrical conductivity (or resistance) of the layer of anisotropic material in directions that are parallel to the front face. For example, the resistance of the layer of anisotropic material in directions parallel to the front face may be less than the first resistance. In some preferred embodiments, the resistance in the parallel directions is less than half of the first resistance or less than 10% of the first resistance. For example, the resistance of the layer of anisotropic material in directions that are parallel to the front face may be less than: 75%, 50%, 40%, 30%, 20%, 10%, 5%, 1%, 0.5%, or even less than 0.1% of the first resistance.

In some embodiments (e.g., when the layer of anisotropic material is a sheet of pyrolytic graphite), the layer of anisotropic material has both anisotropic electrical properties and anisotropic thermal properties.

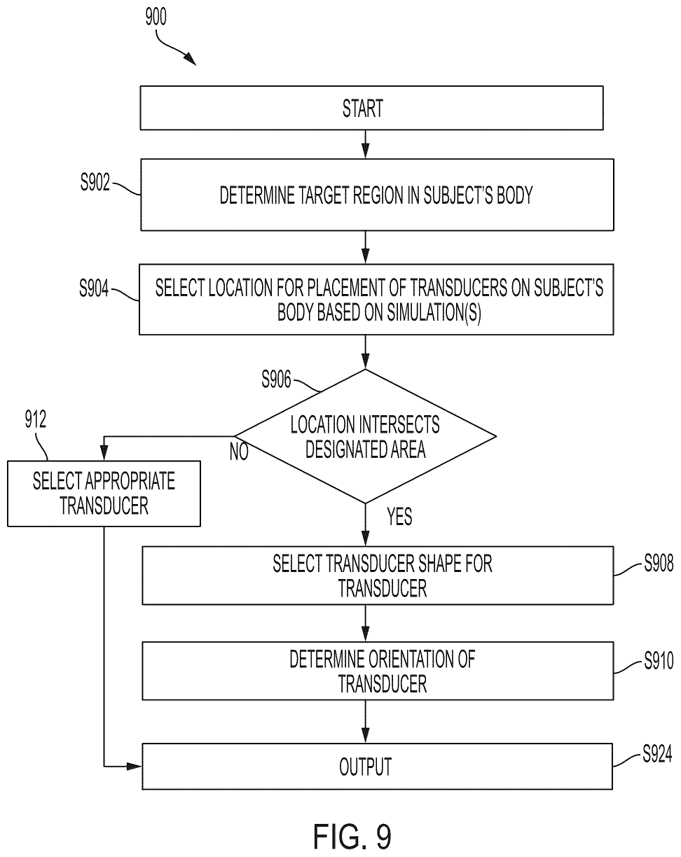

FIG. 8 depicts an example computer apparatus for use with the embodiments herein. As an example, the apparatus 800 may be a computer to implement certain techniques disclosed herein, such as selecting transducer locations for delivering TTFields to a subject as discussed with respect to FIG. 9 below. As an example, the apparatus 800 may be a controller apparatus to apply alternating electric fields for the embodiments herein. For example, the apparatus 800 may be used as the controller 710 of FIG. 7. The apparatus 800 may include one or more processors 802, memory 804, one or more input devices (not shown, but depicted as input 808), and one or more output devices 806.

The memory 804 is accessible by the one or more processors 802 so that the one or more processors 802 can read information from and write information to the memory 804. The memory 804 may store instructions that when executed by the one or more processors 802 implement one or more embodiments of the present disclosure. For example, the apparatus 800 may include one or more processors and memory accessible by the one or more processors, where the memory stores instructions that when executed by the one or more processors, cause the apparatus 800 to perform operations to implement one or more embodiments of the present disclosure.

In one example, based on input 808, the one or more processors 802 may generate control signals to control the voltage generator to implement an embodiment of the invention. The input 808 may be user input, sensor input, or input from another computer in communication with the apparatus 800. The input 808 may be received in conjunction with one or more input devices (not shown) of the apparatus 800. The output devices 806 may provide the status of the operation of the invention, such as transducer selection, voltages being generated, and other operational information. The output devices 806 may provide visualization data.

FIG. 9 is a flowchart depicting an example method 900 of determining the shape and placement of transducers on a subject's body for applying TTFields. As illustrated, the method 900 may begin at step S902 with determining a target region in the subject's body. This may be accomplished by, for example, analyzing one or more sets of image data (e.g., magnetic resonance imaging (MRI) data, computer tomographic (CT) data, etc.) to determine an approximate location and/or 3D volume of the target (e.g., tumor) and/or target region in the subject's body.

At step S904, the method 900 includes selecting a location on the subject's body for placement of transducers based on one or more simulations of an electric field distribution through the target region in the subject's body. This may involve, for example, performing one or more simulations (using a simulation algorithm) of the expected electric field distribution through the target region of the subject's body based on image data associated with the subject's body. More particularly, the determination may be made by comparing simulations for different possible transducer location pairs, ranking the results, and recommending a pair of transducer locations based on expected electric field distributions through the target region.

At step S906, the method 900 includes determining if the location intersects a designated area of the subject's body. The designated area may be, for example, a breast or a chemotherapy port. The breast area may be identified, for example, via an image processing module identifying landmarks (e.g., anatomical features and/or devices) depicted in one or more images included with image data of the subject's body. The image processing module may use one or more object identification and/or tracking algorithms to determine/detect the locations of one or more landmarks. In another example, the breast area may be identified based on user inputs including, for example, an indication of the presence and/or approximate location of a chemotherapy port in the area, body measurements (e.g., breast size measurements), an indication of the presence and/or approximate location of a nipple, and others. An area around a chemotherapy port may similarly be designated.

At step S908, the method 900 includes selecting a transducer shape for the transducer based on the designated area on the subject's body. Selecting the transducer shape may involve, for example, determining at step S906 whether the location overlaps at least a nipple of the breast area or the location overlaps a chemotherapy port. The selected shape may include a shape as described above. In some embodiments, the method 900 may include selecting the particular shape based on factors such as, for example, the relative size and position of the overlapping portion of a breast, a nipple, or a chemotherapy port. The selected transducer shape may be a shape other than a default shape. The selected transducer shape may be a shape as described herein such that the transducer is capable of being deformed from being substantially planar to being substantially non-planar (e.g., being substantially shaped as a truncated elliptical paraboloid, a truncated oblique cone, a truncated cone, or the like).

In an example, at step S910 the method 900 may optionally further include determining an orientation of the transducer at the location of the subject's body to prevent the transducer from covering at least a portion of the designated area (e.g., a nipple or a chemotherapy port in the designated area).

At step S912, upon determining that the location does not overlap a designated area, another transducer shape (e.g., a default transducer shape) may be selected at step S912 for the transducer. As an example, a default transducer shape may be a rectangular, substantially rectangular with rounded corners, circular, oval, ovaloid, ovoid, or an elliptical shape.

At step S924, the method 900 includes outputting the recommended transducer shape and location to a user (e.g., via an output on a user interface). Step S924 may also include outputting a recommended orientation of the transducer to the user. The outputs may be in the form of visual notifications for transducer array placement. That is, the one or more recommended placement positions for the transducer array and a breast, a nipple, and/or a chemotherapy port may be displayed to the user. The notification may visually instruct the user where to place a transducer array to 1) avoid a nipple or a chemotherapy port on the subject's body, and 2) receive an optimized electric field applied to the target region.

While an order of operations is indicated in FIG. 9 for illustrative purposes, the timing and ordering of such operations may vary where appropriate without negating the purpose and advantages of the examples set forth in detail throughout the remainder of this disclosure.

### ILLUSTRATIVE EMBODIMENTS

The invention includes other illustrative embodiments (“Embodiments”) as follows.

Embodiment 1. A transducer apparatus for delivering tumor treating fields to a subject's body, the transducer apparatus comprising a substrate; and an array of at least one electrode disposed on the substrate, the array configured to be positioned over the subject's body with a face of the array facing the subject's body; wherein, when viewed from a direction perpendicular to the face of the array, the substrate has a substantially pear-shaped or rounded triangular-shaped surface having an opening located towards a wider portion of the substantially pear-shaped or rounded triangular-shaped surface, and no electrodes are in the opening, wherein the transducer apparatus is substantially planar, wherein the transducer apparatus is capable of being deformed from being substantially planar to being non-planar and substantially shaped as a truncated elliptical paraboloid or truncated oblique cone, wherein when the transducer apparatus is substantially shaped as a truncated elliptical paraboloid or truncated oblique cone, a substantially circular opening is formed by the opening at a truncated portion of the truncated elliptical paraboloid or truncated oblique cone.

Embodiment 2: The transducer apparatus of Embodiment 1, wherein when viewed from a direction perpendicular to the face of the array and when the transducer apparatus is substantially planar, the transducer apparatus comprises a first end portion separated from a second end portion by a gap, wherein when the transducer apparatus is substantially shaped as a truncated elliptical paraboloid or truncated oblique cone, the first end portion abuts, touches, or overlaps the second end portion.

Embodiment 2A: The transducer apparatus of Embodiment 2, wherein the first end portion has a first edge and a second edge, the first edge defining a portion of an exterior edge of the substantially pear-shaped or rounded triangular-shaped surface, the second edge defining a portion of the opening; and wherein the second end portion has a first edge and a second edge, the first edge defining a portion of the exterior edge of the substantially pear-shaped or rounded triangular-shaped surface, the second edge defining a portion of the opening.

Embodiment 2B: The transducer apparatus of Embodiment 2, wherein the first end portion and the second end portion each include a portion of the array of at least one electrode.

Embodiment 2C: The transducer apparatus of Embodiment 2, wherein only one of the first end portion or the second end portion includes a portion of the array of at least one electrode.

Embodiment 5D: The transducer apparatus of Embodiment 2, wherein the first end portion and the second end portion are part of a single continuous substrate.

Embodiment 5E: The transducer apparatus of Embodiment 2, wherein the first end portion and the second end portion are located on two separate discontinuous sections of substrate.

Embodiment 3: The transducer apparatus of Embodiment 1, wherein when viewed from a direction perpendicular to the face of the array and when the transducer apparatus is substantially planar, the substrate includes a slit between an exterior edge of the substrate and an edge of the opening, wherein the slit separates a first end portion of the transducer apparatus from a second end portion of the transducer apparatus, wherein when the transducer apparatus is substantially shaped as a truncated elliptical paraboloid or truncated oblique cone, the first end portion overlaps the second end portion.

Embodiment 4: The transducer apparatus of Embodiment 1, wherein, when viewed from a direction perpendicular to the face of the array and when the transducer apparatus is substantially planar, the substrate has at least one concave edge defining the opening between two opposing sides of the substrate, and the opening defines a substantially C-shaped surface at the wider portion of the substantially pear-shaped or rounded triangular-shaped surface.

Embodiment 5: The transducer apparatus of Embodiment 4, wherein the substrate has reflectional symmetry.

Embodiment 6: The transducer apparatus of Embodiment 4, wherein a gap defined by the substantially C-shaped surface is situated on one side of the substrate and is defined by a centerline running through a longest dimension of the substrate and through a center of the gap.

Embodiment 7: The transducer apparatus of Embodiment 1, further comprising a second substrate separate from the substrate; wherein the second substrate is substantially C-shaped, wherein when the transducer apparatus is substantially shaped as a truncated elliptical paraboloid or truncated oblique cone, the substantially circular opening is defined by the opening and the substantially C-shaped second substrate; and, optionally, wherein a second array of at least one electrode is disposed on the second substrate, the second array configured to be positioned over the subject's body with a face of the second array facing the subject's body.

Embodiment 8: The transducer apparatus of Embodiment 7, wherein when the transducer apparatus is substantially shaped as a truncated elliptical paraboloid or truncated oblique cone, each end portion of the substantially C-shaped second substrate overlaps separate portions of the substrate.

Embodiment 9: The transducer apparatus of Embodiment 7, wherein the array and the second array are electrically connected.

Embodiment 10: The transducer apparatus of Embodiment 1, wherein the substrate is adapted to be positioned on or around an anatomical feature of a subject.

Embodiment 11: The transducer apparatus of Embodiment 10, wherein the anatomical feature is a breast.

Embodiment 12: The transducer apparatus of Embodiment 1, wherein the substantially circular opening coincides with a first location on a subject, and wherein the first location comprises a nipple, and no electrodes of the transducer array are located over the nipple.

Embodiment 13: The transducer apparatus of Embodiment 1, further comprising a layer of anisotropic material on a skin-facing side of the array.

Embodiment 14: The transducer apparatus of Embodiment 13, wherein the anisotropic material is a sheet of graphite.

Embodiment 14A: The transducer apparatus of Embodiment 13, wherein the layer of anisotropic material is a sheet of pyrolytic graphite, graphitized polymer film, or graphite foil made from compressed high purity exfoliated mineral graphite.

Embodiment 14B: A method of using the apparatus of Embodiment 1, comprising applying the substrate on or around an anatomical feature of a subject's body such that the substantially circular opening coincides with a first location on the subject's body; and generating an electric field from the array of electrodes.

Embodiment 14C: The method of Embodiment 14B, wherein the first location comprises a nipple, and no electrodes of the array are located over the nipple.

Embodiment 14D: A transducer apparatus for delivering tumor treating fields to a subject's body, the transducer apparatus comprising: a substrate; and an array of at least one electrode disposed on the substrate, the array configured to be positioned over the subject's body with a face of the array facing the subject's body; wherein, when viewed from a direction perpendicular to the face of the array, the substrate has a substantially pear-shaped or rounded triangular-shaped surface, the substrate has two ends defining a gap between two opposing sides of the substrate, the gap located closer to a wider portion than a narrower portion of the substantially pear-shaped or rounded triangular-shaped surface, and no electrodes are in the gap, wherein the transducer apparatus is substantially planar, wherein the transducer apparatus is capable of being deformed from being substantially planar to being non-planar and substantially shaped as a truncated elliptical paraboloid or truncated oblique cone, wherein when the transducer apparatus is substantially shaped as a truncated elliptical paraboloid or truncated oblique cone, a substantially circular opening is formed at a truncated portion of the truncated elliptical paraboloid or truncated oblique cone by removing the gap between two opposing sides of the substrate.

Embodiment 14E: A computer-implemented method to determine a shape and placement of transducers on a subject's body for applying tumor treating fields, the computer comprising one or more processors and memory accessible by the one or more processors, the memory storing instructions that when executed by the one or more processors cause the computer to perform the method, the method comprising: determining a target region in the subject's body to apply tumor treating fields; selecting a first location on the subject's body for placement of a first transducer apparatus and a second location on the subject's body for placement of a second transducer apparatus, based on one or more simulations of an electric field distribution through the target region in the subject's body; selecting a first transducer apparatus based on the first location intersecting a breast area of the subject's body; selecting a second transducer apparatus for the second location; and outputting the first transducer apparatus, the first location, the second transducer apparatus, and the second location to a user, wherein, when viewed from a direction perpendicular to the face of the first transducer apparatus, the first transducer apparatus has a substantially pear-shaped or rounded triangular-shaped surface having an opening located towards a wider portion of the substantially pear-shaped or rounded triangular-shaped surface, wherein the first transducer apparatus is capable of being deformed from being substantially planar to being non-planar and substantially shaped as a truncated elliptical paraboloid or truncated oblique cone, wherein when the first transducer apparatus is substantially shaped as a truncated elliptical paraboloid or truncated oblique cone, a substantially circular opening is formed at a truncated portion of the truncated elliptical paraboloid or truncated oblique cone.

Embodiment 15: A transducer apparatus for delivering tumor treating fields to a subject's body, the transducer apparatus comprising a substrate; and an array of at least one electrode disposed on the substrate, the array configured to be positioned over the subject's body with a face of the array facing the subject's body; wherein the transducer apparatus is substantially non-planar, wherein the transducer apparatus is substantially shaped as a truncated elliptical paraboloid, truncated oblique cone, or truncated cone, and a substantially circular opening is formed by an opening at a truncated portion of the truncated elliptical paraboloid, truncated oblique cone, or truncated cone.

Embodiment 16: The transducer apparatus of Embodiment 15, wherein the substrate is adapted to be positioned on or around an anatomical feature of a subject.

Embodiment 16A: The transducer apparatus of Embodiment 16, wherein the anatomical feature is a breast.

Embodiment 17: The transducer apparatus of Embodiment 15, wherein the substantially circular opening coincides with a first location on a subject, wherein the first location comprises a nipple, and no electrodes of the transducer array are located over the nipple.

Embodiment 18: The transducer apparatus of Embodiment 15, further comprising a layer of anisotropic material on a skin-facing side of the array.

Embodiment 19: The transducer apparatus of Embodiment 18, wherein the anisotropic material is a sheet of graphite.

Embodiment 19A: The transducer apparatus of Embodiment 18, wherein the layer of anisotropic material is a sheet of pyrolytic graphite, graphitized polymer film, or graphite foil made from compressed high purity exfoliated mineral graphite.

Embodiment 20: A computer-implemented method to determine a shape and placement of transducers on a subject's body for applying tumor treating fields, the computer comprising one or more processors and memory accessible by the one or more processors, the memory storing instructions that when executed by the one or more processors cause the computer to perform the method, the method comprising: determining a target region in the subject's body to apply tumor treating fields; selecting a first location on the subject's body for placement of a first transducer apparatus and a second location on the subject's body for placement of a second transducer apparatus, based on one or more simulations of an electric field distribution through the target region in the subject's body; selecting a first transducer apparatus based on the first location intersecting a breast area of the subject's body; selecting a second transducer apparatus for the second location; and outputting the first transducer apparatus, the first location, the second transducer apparatus, and the second location to a user, wherein the first transducer apparatus is substantially non-planar, wherein the first transducer apparatus is substantially shaped as a truncated elliptical paraboloid, truncated oblique cone, or truncated cone, and a substantially circular opening is formed at a truncated portion of the truncated elliptical paraboloid, truncated oblique cone, or truncated cone.

Embodiment 21: A method of administering tumor treating fields (TTFields) to a subject, the method comprising: determining a target region in the subject's body to apply tumor treating fields; selecting a location on the subject's body for placement of a transducer apparatus based on one or more simulations of an electric field distribution through the target region in the subject's body; selecting a transducer apparatus based on the location intersecting a breast area of the subject's body, the transducer apparatus comprising a first end portion separated from a second end portion by a gap, wherein when the transducer apparatus is substantially shaped as a truncated elliptical paraboloid, truncated oblique cone, or truncated cone, the first end portion abuts, touches, or overlaps the second end portion; applying the transducer apparatus to the location on the subject's body intersecting the breast area of the subject's body; and overlapping the first end portion and the second end portion to obtain a 3-D structure or a non-planar transducer structure, wherein a substantially circular opening is formed at a truncated portion of the truncated elliptical paraboloid, truncated oblique cone, or truncated cone.

Embodiment 21A: The method of Embodiment 21, further comprising: delivering alternating electric fields to the subject via at least one pair of transducer apparatuses, wherein the at least one pair of transducer apparatuses comprises the transducer apparatus on the location intersecting the breast area of the subject's body.

Embodiment 22: A method of positioning a transducer array over a contoured surface of a subject, the method comprising: determining a target region in the subject's body to apply tumor treating fields; selecting a location on the subject's body for placement of a transducer apparatus based on one or more simulations of an electric field distribution through the target region in the subject's body; selecting a transducer apparatus based on the location intersecting a breast area of the subject's body, the transducer apparatus comprising a first end portion separated from a second end portion by a gap, wherein when the transducer apparatus is substantially shaped as a truncated elliptical paraboloid, truncated oblique cone, or truncated cone, the first end portion abuts, touches, or overlaps the second end portion; applying the transducer apparatus to the location on the subject's body intersecting the breast area of the subject's body; and overlapping the first end portion and the second end portion to obtain a 3-D structure or a non-planar transducer structure, wherein a substantially circular opening is formed at a truncated portion of the truncated elliptical paraboloid, truncated oblique cone, or truncated cone.

Embodiment 22A: The method of Embodiment 22, further comprising: delivering alternating electric fields to the subject via at least one pair of transducer apparatuses, wherein the at least one pair of transducer apparatuses comprises the transducer apparatus on the location intersecting the breast area of the subject's body.

Optionally, for each embodiment described herein, the voltage generation components supply the transducers with an electrical signal having an alternating current waveform at frequencies in a range from about 50 kHz to about 1 MHz, or from about 100 kHz to about 500 kHz, and appropriate to deliver TTFields treatment to the subject's body.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention not be limited to the described embodiments, but that it has the full scope defined by the language of the following claims, and equivalents thereof.

What is claimed is:

1. A transducer apparatus for delivering tumor treating fields to a subject's body, the transducer apparatus comprising:
a substrate; and
an array of at least one electrode disposed on the substrate, the array configured to be positioned over the subject's body with a face of the array facing the subject's body;
wherein, when viewed from a direction perpendicular to the face of the array,
the substrate has a substantially pear-shaped or rounded triangular-shaped surface having an opening located towards a wider portion of the substantially pear-shaped or rounded triangular-shaped surface, and
no electrodes are in the opening,
wherein the transducer apparatus is substantially planar,
wherein the transducer apparatus is capable of being deformed from being substantially planar to being non-planar and substantially shaped as a truncated elliptical paraboloid or truncated oblique cone,
wherein when the transducer apparatus is substantially shaped as a truncated elliptical paraboloid or truncated oblique cone, a substantially circular opening is formed by the opening at a truncated portion of the truncated elliptical paraboloid or truncated oblique cone.

2. The transducer apparatus of claim 1,
wherein when viewed from a direction perpendicular to the face of the array and when the transducer apparatus is substantially planar, the transducer apparatus comprises a first end portion separated from a second end portion by a gap,
wherein when the transducer apparatus is substantially shaped as a truncated elliptical paraboloid or truncated oblique cone, the first end portion abuts, touches, or overlaps the second end portion.

3. The transducer apparatus of claim 1,
wherein when viewed from a direction perpendicular to the face of the array and when the transducer apparatus is substantially planar, the substrate includes a slit between an exterior edge of the substrate and an edge of the opening,
wherein the slit separates a first end portion of the transducer apparatus from a second end portion of the transducer apparatus,
wherein when the transducer apparatus is substantially shaped as a truncated elliptical paraboloid or truncated oblique cone, the first end portion overlaps the second end portion.

4. The transducer apparatus of claim 1,
wherein, when viewed from a direction perpendicular to the face of the array and when the transducer apparatus is substantially planar,
the substrate has at least one concave edge defining the opening between two opposing sides of the substrate, and
the opening defines a substantially C-shaped surface at the wider portion of the substantially pear-shaped or rounded triangular-shaped surface.

5. The transducer apparatus of claim 4, wherein the substrate has reflectional symmetry.

6. The transducer apparatus of claim 4, wherein a gap defined by the substantially C-shaped surface is situated on one side of the substrate and is defined by a centerline running through a longest dimension of the substrate and through a center of the gap.

7. The transducer apparatus of claim 1, further comprising:
a second substrate separate from the substrate;
wherein the second substrate is substantially C-shaped,
wherein when the transducer apparatus is substantially shaped as a truncated elliptical paraboloid or truncated oblique cone, the substantially circular opening is defined by the opening and the substantially C-shaped second substrate; and, optionally,
wherein a second array of at least one electrode is disposed on the second substrate, the second array configured to be positioned over the subject's body with a face of the second array facing the subject's body.

8. The transducer apparatus of claim 7, wherein when the transducer apparatus is substantially shaped as a truncated elliptical paraboloid or truncated oblique cone, each end portion of the substantially C-shaped second substrate overlaps separate portions of the substrate.

9. The transducer apparatus of claim 7, wherein the array and the second array are electrically connected.

10. The transducer apparatus of claim 1, wherein the substrate is adapted to be positioned on or around an anatomical feature of a subject.

11. The transducer apparatus of claim 10, wherein the anatomical feature is a breast.

12. The transducer apparatus of claim 1, wherein the substantially circular opening coincides with a first location on a subject, wherein the first location comprises a nipple, and no electrodes of the transducer array are located over the nipple.

13. The transducer apparatus of claim 1, further comprising a layer of anisotropic material on a skin-facing side of the array.

14. The transducer apparatus of claim 13, wherein the anisotropic material is a sheet of graphite.

15. A transducer apparatus for delivering tumor treating fields to a subject's body, the transducer apparatus comprising:
a substrate; and
an array of at least one electrode disposed on the substrate, the array configured to be positioned over the subject's body with a face of the array facing the subject's body;
wherein the transducer apparatus is substantially non-planar,
wherein the transducer apparatus is substantially shaped as a truncated elliptical paraboloid, truncated oblique cone, or truncated cone, and a substantially circular opening is formed by an opening at a truncated portion of the truncated elliptical paraboloid, truncated oblique cone, or truncated cone.

16. The transducer apparatus of claim 15, wherein the substrate is adapted to be positioned on or around an anatomical feature of a subject.

17. The transducer apparatus of claim 15, wherein the substantially circular opening coincides with a first location on a subject, wherein the first location comprises a nipple, and no electrodes of the transducer array are located over the nipple.

18. The transducer apparatus of claim 15, further comprising a layer of anisotropic material on a skin-facing side of the array.

19. The transducer apparatus of claim 18, wherein the anisotropic material is a sheet of graphite.

20. A computer-implemented method to determine a shape and placement of transducers on a subject's body for applying tumor treating fields, the computer comprising one or more processors and memory accessible by the one or more processors, the memory storing instructions that when executed by the one or more processors cause the computer to perform the method, the method comprising:
determining a target region in the subject's body to apply tumor treating fields;
selecting a first location on the subject's body for placement of a first transducer apparatus and a second location on the subject's body for placement of a second transducer apparatus, based on one or more simulations of an electric field distribution through the target region in the subject's body;
selecting a first transducer apparatus based on the first location intersecting a breast area of the subject's body;
selecting a second transducer apparatus for the second location; and
outputting the first transducer apparatus, the first location, the second transducer apparatus, and the second location to a user,
wherein the first transducer apparatus is substantially non-planar,
wherein the first transducer apparatus is substantially shaped as a truncated elliptical paraboloid, truncated oblique cone, or truncated cone, and a substantially circular opening is formed at a truncated portion of the truncated elliptical paraboloid, truncated oblique cone, or truncated cone.
